(19) United States
(12) Patent Application Publication
(10) Pub. No.: US 2025/0327889 A1
(43) Pub. Date: Oct. 23, 2025

(54) MAGNETIC RESONANCE IMAGE RECONSTRUCTION WITH DEEP LEARNING-BASED OUTER VOLUME REMOVAL

(71) Applicant: Regents of the University of Minnesota, Minneapolis, MN (US)

(72) Inventors: Mehmet Akçakaya, Minneapolis, MN (US); Merve Gulle, Minneapolis, MN (US)

(21) Appl. No.: 19/182,191

(22) Filed: Apr. 17, 2025

Related U.S. Application Data
(60) Provisional application No. 63/635,083, filed on Apr. 17, 2024.

(51) Int. Cl. G01R 33/56; A61B 5/00; G01R 33/48; G06T 11/00

(52) U.S. Cl. CPC G01R 33/5608; A61B 5/0044; G01R 33/4818; G06T 11/005; G06T 11/006; G06T 2210/41; G06T 2211/441

(57) ABSTRACT

A method for magnetic resonance image reconstruction with outer volume removal includes accessing timeframes of k-space data acquired using time-interleaved undersampling patterns in k-space. A composite image is generated from the k-space data by combining timeframes of the k-space data. A machine learning model—trained on training data to extract ghosting artifact signal components from a magnetic resonance image—is used to generate a ghosting artifact image by inputting the composite image data to the machine learning model. Outer volume signals are estimated by subtracting the ghosting artifact image from the composite image. Outer volume removed k-space data are generated by removing the outer volume signals from the k-space data. One or more images are reconstructed from the outer volume removed k-space data.

### CROSS-REFERENCE TO RELATED APPLICATIONS

This application claims the benefit of U.S. Provisional Patent Application Ser. No. 63/635,083, filed on Apr. 17, 2024, and entitled “MAGNETIC RESONANCE IMAGE RECONSTRUCTION WITH DEEP LEARNING-BASED OUTER VOLUME SUPPRESSION,” which is herein incorporated by reference in its entirety.

### STATEMENT OF FEDERALLY SPONSORED RESEARCH

This invention was made with government support under HL153146 and EB027061 awarded by the National Institutes of Health. The government has certain rights in the invention.

### BACKGROUND

Real-time dynamic magnetic resonance imaging (MRI) uses rapid snapshot acquisitions to visualize dynamic processes. These techniques are particularly important for cardiac applications. For instance, real-time cine imaging allows free-breathing ECG-free quantification of myocardial function for patients with impaired breath-hold capacity or arrhythmia, while real-time late gadolinium enhancement (LGE) imaging allows for free-breathing quantification of the cardiac scar. However, current real-time cardiac MRI techniques using parallel imaging often have limited acceleration leading to low spatiotemporal resolution. Alternatively, spatiotemporal regularization has been used for higher accelerations for real-time cine MRI, but these risk temporal blurring while also incurring high computational complexity. Thus, reconstruction methods that allow higher acceleration rates without temporal regularization are desirable.

The major impediment towards achieving higher acceleration rates in parallel imaging reconstruction of the real-time cardiac MRI data is the large outer volume of extra-cardiac tissue that aliases into the heart. One method to tackle this challenge in broader cardiac applications has been the use of outer volume suppression pulses/modules, but these have not found utility in real-time MRI due to their lengths and the disruption to steady-state.

### SUMMARY OF THE DISCLOSURE

According to an aspect of the present disclosure, a method for reconstructing an image of a subject from k-space data acquired with a magnetic resonance imaging (MRI) system is provided. The method includes accessing k-space data with a computer system, wherein the k-space data were acquired from a subject using an MRI system, wherein the k-space data include timeframes of k-space data acquired using time-interleaved undersampling patterns in k-space. The method also includes generating composite image data from the k-space data using the computer system to combine timeframes of the k-space data. The method further includes accessing a machine learning model with the computer system, wherein the machine learning model has been trained on training data to extract ghosting artifact signal components from a magnetic resonance image. The method additionally includes generating a ghosting artifact image by inputting the composite image data to the machine learning model using the computer system, generating the ghosting artifact image as an output, wherein the ghosting artifact image depicts ghosting artifacts extracted from the composite image data. The method also includes estimating outer volume signals using the computer system to subtract the ghosting artifact image from the composite image data. The method further includes generating outer volume removed k-space data using the computer system to remove the outer volume signals from the k-space data. The method additionally includes reconstructing an image from the outer volume removed k-space data using the computer system.

According to another aspect of the present disclosure, a method for generating outer volume removed calibration data for use in magnetic resonance imaging (MRI) is provided. The method includes accessing k-space data with a computer system, wherein the k-space data were acquired from a subject using an MRI system, wherein the k-space data include timeframes of k-space data acquired using time-interleaved undersampling patterns in k-space. The method also includes generating composite image data from the k-space data using the computer system to combine timeframes of the k-space data. The method further includes estimating outer volume signals from the composite image data. The method additionally includes generating outer volume removed calibration data using the computer system to remove the outer volume signals from the composite image data. The method also includes storing the outer volume removed data with the computer system.

According to another aspect of the present disclosure, a method for reconstructing an image of a subject from k-space data acquired with a magnetic resonance imaging (MRI) system is provided. The method includes accessing k-space data with a computer system, wherein the k-space data were acquired from a heart of a subject with an MRI system using a pulse sequence, wherein the k-space data include one timeframe of k-space data that samples a central region of k-space and at least one timeframe of k-space data that samples peripheral regions of k-space during dead time of a pulse sequence during which the heart is moving. The method also includes reconstructing a first image from the k-space data using a deep learning image reconstruction that is trained on training data to remove image artifacts. The method further includes generating composite image data from the k-space data using the computer system to combine timeframes of the k-space data. The method additionally includes estimating outer volume signals using the computer system to subtract the first image from the composite image data. The method also includes generating outer volume removed k-space data using the computer system to remove the outer volume signals from the k-space data. The method further includes reconstructing a second image from the outer volume removed k-space data using the computer system.

### BRIEF DESCRIPTION OF THE DRAWINGS

FIG. 1 is an example decomposition of a composite real-time cine cardiac MR image into various components. For simpler visualization, the heart is depicted as the only moving object, while surrounding tissues are treated as stationary, and R=4 is used. Both the moving components and stationary components of each timeframe contribute to the composite image as foldovers with distinct modulation coefficients due to the shifted phase encoding lines across timeframes. The moving components add constructively at the true heart location, creating a temporally averaged representation, while their summation at the other foldover locations leads to a pseudo-periodic ghosting artifact due to cardiac motion between timeframes and the distinct modulation coefficients. On the other hand, the stationary components add constructively at the central foldover while canceling out at the other foldover locations.

FIGS. 2A-2E illustrate and example reconstruction pipeline that can be used with the outer volume removal techniques described in the present disclosure. FIG. 2A shows a process for DL-based ghosting detection: A ResNet with 15 residual blocks (RBs) takes four adjacent composite images, concatenating their real and imaginary components into eight input channels (Cin=8), and estimates the ghosting artifacts for the corresponding time frame t₀, producing eight output channels (Cout=8). The stationary background coil images, x_{background}, are then obtained by subtracting the detected ghosting artifacts from the composite images of the target time frame. FIG. 2B shows an example physics-driven DL-based unrolled network: The network includes 35 unrolls, where each iteration block contains one ResNet and one data fidelity (DF) block. The ResNets (Cin=2, Cout=2, 15 residual blocks) perform the proximal operation for the regularizer on the previous DF block output, and share the same network weights, θ. The DF block takes the zerofilled image (x₀), the last ResNet output (zₖ), sensitivity maps, and the acceleration mask, producing a data-consistent image as output using conjugate gradient. FIG. 2C shows an example ResNet structure used in both the ghosting detection network and the proximal operators of the unrolled network. FIG. 2D shows an example outer volume removal and PD-DL-based reconstruction: The background image is masked using the outer volume removal mask (m_{OVR}) and transformed into k-space via the Fourier transform (F^{Ω}^{ₜ}), then subtracted from the acquired signal (y^{Ω}^{ₜ}). The outer volume removed k-space signal

$${y}_{OVR}^{{Ω}_{t}}$$

is mapped back to the image domain using the adjoint encoding operator (E^{Ω}^{ₜ}) and fed into the unrolled network. The network output exhibits minimal background signal. FIG. 2E shows an example of final image formation by combining the masked background with the reconstruction.

FIG. 3 is a flowchart setting forth the steps of an example method for reconstructing an image of a subject using an outer volume removed image reconstruction pipeline.

FIG. 4A illustrates an example composite k-space data set generated from timeframes of time-interleaved k-space data.

FIG. 4B illustrates another example composite k-space data set generated from timeframes of time-interleaved k-space data.

FIG. 4C is an example of timeframes of k-space data acquired using a time-interleaved undersampling of k-space.

FIG. 5 is an example of a composite image and a corresponding outer volume mask generated from outer volume signals estimated from the composite image.

FIG. 6 is an example of a timeframe of outer volume removed k-space data.

FIG. 7 is an example of an outer volume removed composite image.

FIG. 8 is an illustration of an example overall pipeline for reconstructing dynamic real-time MRI using outer volume subtraction and an unrolled physics-driven deep learning network.

FIGS. 9A-9C illustrate an example PD-DL reconstruction of outer volume removed k-space data using masked sensitivity maps (FIG. 9A), full sensitivity maps (FIG. 9B), and full sensitivity maps with consistency through the loss function in Eqn. (9) (FIG. 9C). The left column shows artifacts, while the middle column exhibits signal loss. The proposed consistency mechanism effectively mitigates both issues, as demonstrated in the right column.

FIG. 10 is a comparative example of coil sensitivity maps generated from calibration data with and without outer volume removal.

FIG. 11 is a comparative example of images reconstructed with no outer volume removal, with outer volume removal and using regular calibration data, and with outer volume removal and using outer volume removed calibration data.

FIG. 12 is a flowchart setting forth the steps of an example method for training a machine learning model to estimate ghosting artifact signal components from a composite image.

FIG. 13 shows example images demonstrating that disclosed methods accurately detects ghosting artifacts, leading to improved outer volume images.

FIG. 14 shows representative reconstructions of real-time cine MRI with baseline TGRAPPA R=4, as well as TGRAPPA, PD-DL without outer volume subtraction, and the proposed method at R=8. While TGRAPPA at R=8 suffers from substantial residual artifacts, PD-DL without outer volume subtraction reconstructions has blurriness and residual artifacts on the myocardium muscle (arrows) and the papillary muscles. The proposed method visibly outperforms both TGRAPPA at the same rate and PD-DL without outer volume subtraction, demonstrating quality comparable to the baseline R=4 image. This includes a clear depiction of blood-myocardium borders and a reduction of spatially varying noise in the baseline images.

FIGS. 15A and 15B show an illustration of a conventional (FIG. 15A) and ECG-triggered (FIG. 15B) acquisition. In the conventional acquisition (FIG. 15A), data are acquired only during the imaging window following preparation pulses. In contrast, the sequence in FIG. 15B continues acquisition during the gap before the next heartbeat, capturing auxiliary data used to estimate the outer volume signal.

FIG. 16 is an illustration of a total window parallel reconstruction image components at R=2 was used. The imaging and auxiliary windows were acquired in different cardiac phases, capturing heart movement while the background remains stationary over a ˜200 ms interval. Each frame was analyzed as a combination of moving (heart) and stationary (background) components. Consequently, the parallel reconstruction of the total window includes a stationary background, a low temporal resolution heart reflecting an average of two cardiac phases, and noise-like ghosting artifacts from cardiac motion occurring between the imaging and auxiliary windows.

FIG. 17 shows a comparison of ZS-SSDU reconstructions with and without outer volume removal. The first row shows the parallel image reconstruction all the acquired data at R=2, along with the estimated outer volume masked from the reconstruction (ghosting artifacts due to cardiac motion indicated by arrows). The second and third rows display the zero-filled images (left) and final reconstructions (right). Without outer volume removal, folding artifacts remain visible; however, using the outer volume removal techniques described in the present disclosure effectively eliminates these artifacts, providing a clearer myocardium border.

FIG. 18 is a block diagram of an example MRI system that can implement some methods described in the present disclosure.

FIG. 19 is a block diagram of an example system for outer volume removed and/or image reconstruction.

FIG. 20 is a block diagram of example components that can implement the system of FIG. 19.

### DETAILED DESCRIPTION

Described here are systems and methods for achieving outer volume removal and reconstructing images of a smaller field-of-view from data acquired from a larger spatial region. For example, the systems and methods can be used to reconstruct images of the heart in cardiac imaging application, the prostate in body imaging applications, the brain in neuroimaging applications, and so on. In these cases, a larger field-of-view is acquired to avoid aliasing artifacts, often leading to inefficiencies.

The performance of parallel imaging, or more generally multicoil image reconstruction, is dictated at least in part by the coil geometry, as evident in the name g-factor (i.e., geometry factor). To date, not much attention has been paid to how calibration data should be used in outer volume removed scenarios. It is contemplated that when regular calibration data (i.e., calibration data including outer volume signals) is used for image reconstruction of outer volume removed data, then the image reconstruction performance will be sub-optimal. It is therefore an advantage of the methods described in the present disclosure to combine an outer volume removal technique with a calibration strategy that also utilizes outer volume removal.

In general, a low temporal resolution, but also low acceleration, composite image is generated, from which unwanted outer volume can be segmented. This outer volume can then be subtracted from both the composite k-space, creating outer volume removed calibration data, and from high temporal resolution high acceleration k-space of interest.

As an example, pseudo-periodic ghosting artifacts arising from the moving tissue in low-temporal resolution composite images can be characterized, and deep learning can be used to remove these artifact signal components from the composite images. Subsequently, the estimated outer volume signals are subtracted from each individual timeframe to eliminate the outer volume signals. These data are then reconstructed using a physics-driven deep learning model, or other suitable image reconstruction technique or model, at high-temporal resolutions.

Let x(t)∈ [symbol] represent the t^{th} complex-valued timeframe of a dynamic MRI sequence, where N is the number of pixels in the spatial plane, which as a non-limiting example may be assumed to be 2D without loss of generality. The acquired k-space data, denoted as y^{Ω}^{ₜ}(t)∈ [symbol] , corresponds to the measurements from k-space locations Ωₜ, with M being the number of acquired k-space points per timeframe. Using a time-interleaved shifted equidistant or uniform undersampling pattern, a fully sampled composite k-space or image with low temporal resolution can be generated by combining R consecutive timeframes into a single merged dataset for acceleration rate R.

The systems and methods described in the present disclosure utilize an analytical perspective on such composite data by treating the images from each timeframe as a combination of moving, x_{moving}(t), and stationary, x_{stationary}(t), components as:

$$x ( t ) = {x}_{moving} ( t ) + {x}_{stationary} ( t ) . \qquad ( 1 )$$

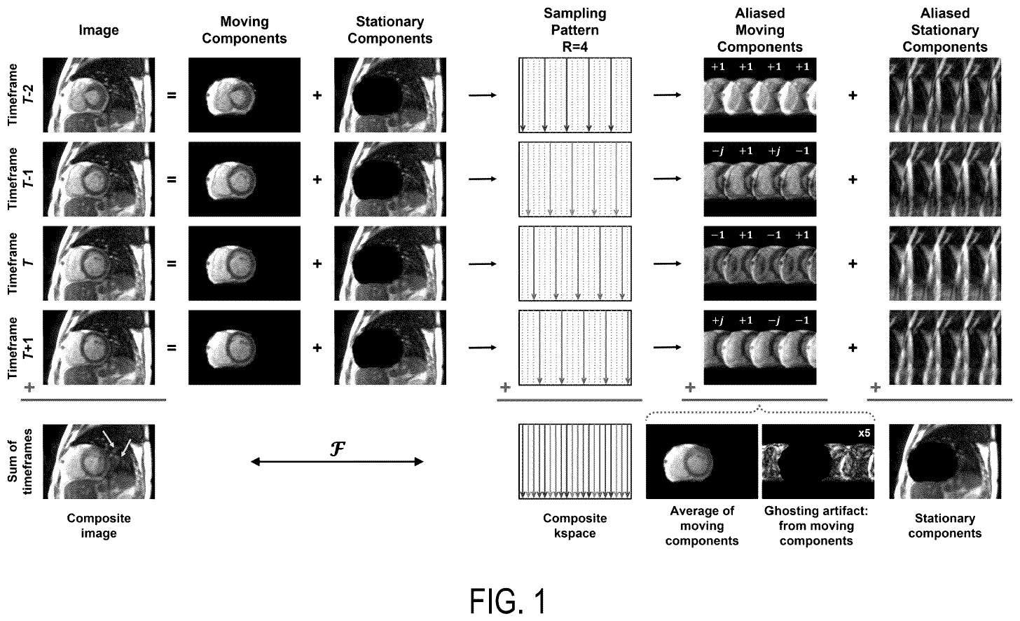

FIG. 1 illustrates this decomposition across different timeframes, where for simplicity, the heart is depicted as the only moving object, while the surrounding tissues are considered stationary across these timeframes. For real-time sequences, these assumptions can hold over the acquisition of R subsequent frames, provided the temporal resolution is sufficiently small, since the respiratory motion is much slower compared to cardiac motion. In this example scenario, each individual timeframe contributes an aliased image of the heart and the stationary background to the composite image. Due to the time-interleaved shifted pattern in the k-space acquisitions, each foldover of the aliased components has a distinct modulation phase. In the composite image, the side foldovers of the aliased background tend to cancel each other out, resulting in a stationary background. Conversely, the foldovers of the aliased heart images align at the true heart location, forming a temporally averaged representation of the heart at its correct position. Finally, other foldovers of the moving components add up to produce a ghosting artifact in the background due to differing modulation phases, which manifests as a pseudo-periodic pattern.

Overall, this formulation allows for the decomposition of the composite image into the combination of temporarily averaged moving components x_{moving}(t), a pseudo-periodic ghosting artifact x_{ghost}(t) due to the moving tissue components, and a stationary background x_{stationary}(t) as:

$${x}_{composite} ( t ) = {\underbrace{{\overline{x}}_{moving} ( t ) + {x}_{ghost} ( t )}}_{moving components} + {\underbrace{{x}_{background} ( t )}}_{stationary components} . \qquad ( 2 )$$

In contrast to the simplified depiction in FIG. 1, moving tissues other than the heart, such as the diaphragm, can move rapidly enough to contribute to such ghosting artifacts. This simplified version is therefore used illustrative purposes here, whereas these three image components can be captured without explicitly delineating boundaries for moving organs.

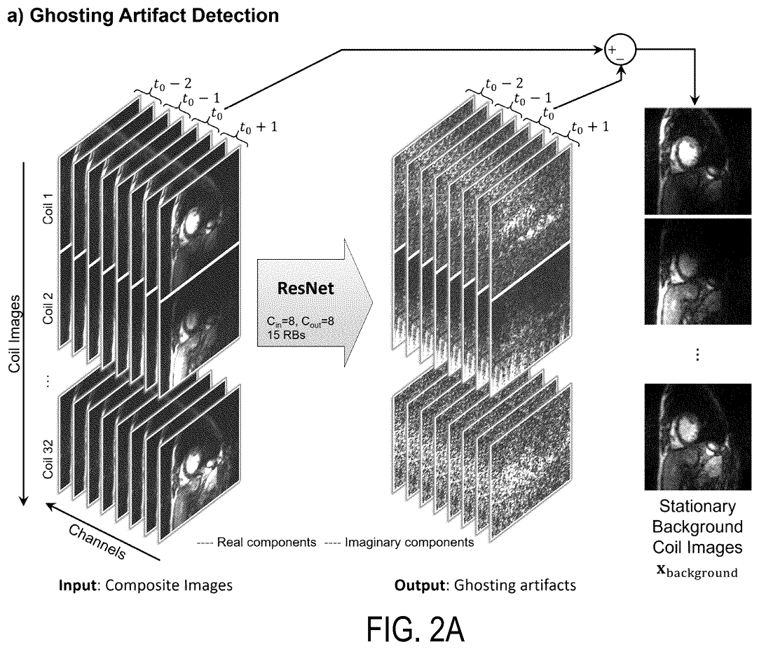

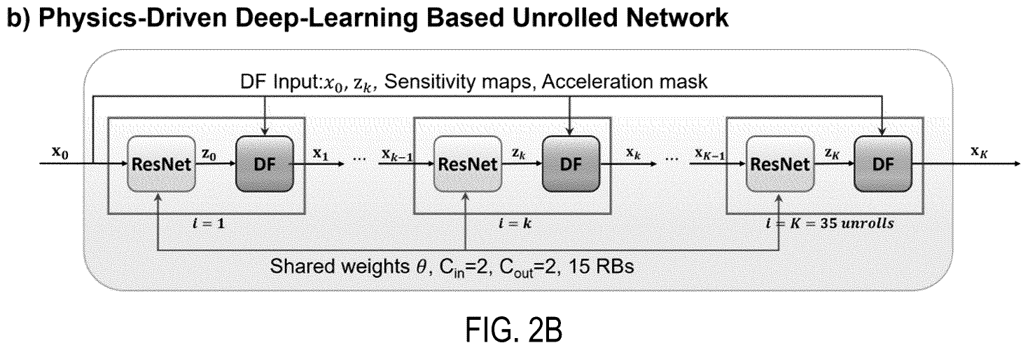

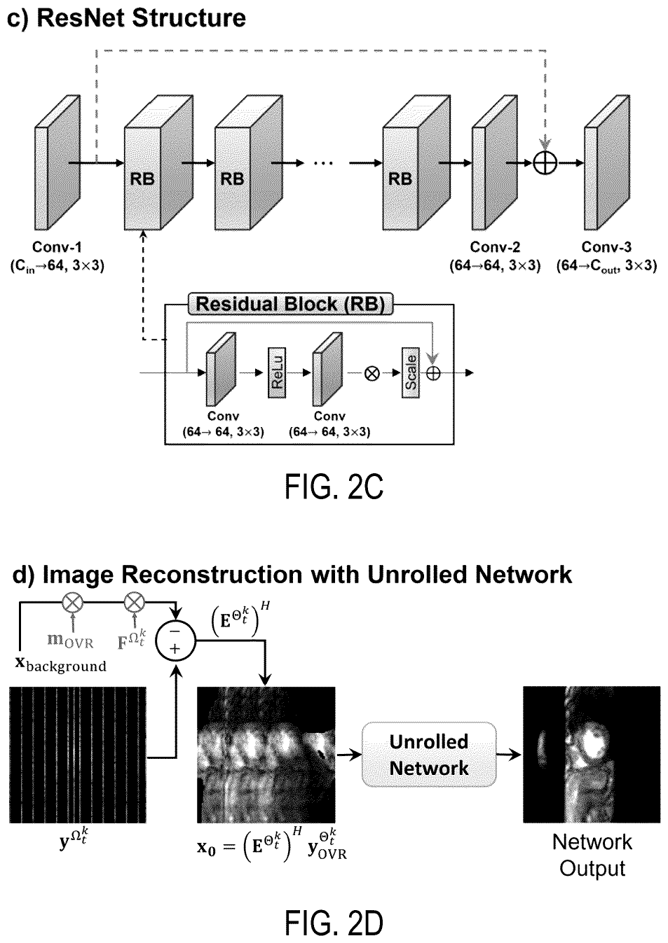

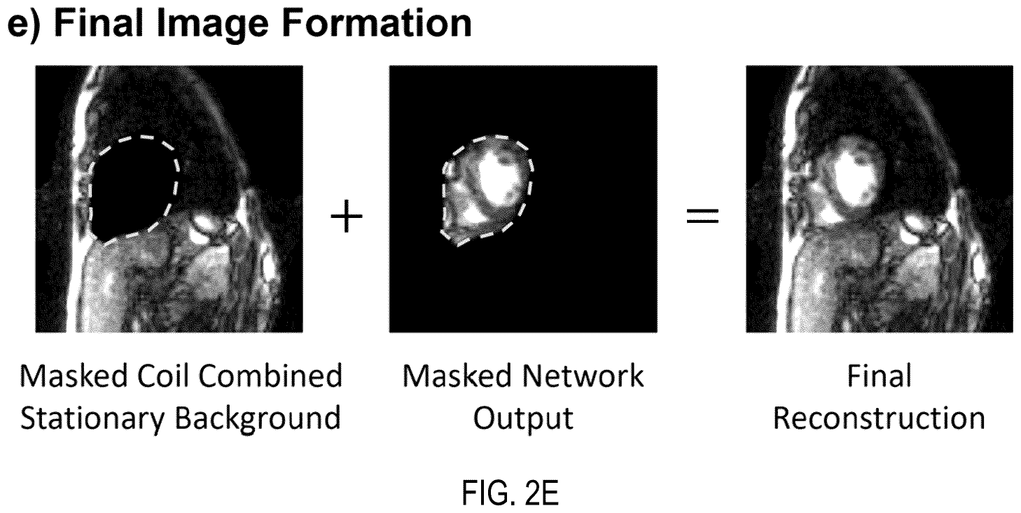

As an illustrative example, let x_{composite}(t₀) denote the composite image of time t₀. To estimate the background of this acquisition x_{background}(t₀) in this illustrative example, a DL-based technique can be used to estimate the motion-related pseudo-periodic ghosting artifacts in the composite images illustrated in FIG. 2A. The network takes four adjacent composite images in channel-wise concatenated form, represented as

$${x}_{composite}^{concat} ( {t}_{0} ) {=}^{△} {\{ {x}_{composite} ( τ ) \}}_{τ = {t}_{0} - 2}^{{t}_{0} + 1} ,$$

covering a wider temporal window to leverage the correlation over timeframes. The network outputs the corresponding ghosting artifacts of each composite image in the output, again in a channel-wise concatenated form

$${x}_{ghost}^{concat} ( {t}_{0} ) {=}^{△} {\{ {x}_{ghost} ( τ ) \}}_{τ = {t}_{0} - 2}^{{t}_{0} + 1} .$$

It is noted that all four images are used for loss calculation, but only the ghosting estimate at t₀ is used for subsequent outer volume removal for the corresponding timeframe.

The network is trained in a supervised manner by minimizing a normalized [symbol] ₂ loss between the network output and the reference ghosting artifacts:

$${min}_{θ} 𝔼 [ {ℒ}_{normalized - {ℓ}_{2}} ( {x}_{ghost - ref}^{concat} ( t ) , {f}_{θ} ( {x}_{composite}^{concat} ( t ) ) ) ] ; \qquad ( 3 )$$

where

$${x}_{ghost - ref}^{concat} ( t )$$

is the concatenated reference ghosting image, and f_{θ}(⋅) is the ghosting detection network with trainable parameters θ. After the estimation of these ghosting artifacts, their contribution is subtracted from the corresponding composite images to obtain clean outer volume background images:

$${x}_{background} ( {t}_{0} ) \approx {x}_{composite} ( {t}_{0} ) - {f}_{θ} ( {x}_{i , composite}^{concat} ( {t}_{0} ) ) {\vert}_{t = {t}_{0}} ; \qquad ( 4 )$$

where

$${f}_{θ} ( {x}_{i , composite}^{concat} ( {t}_{0} ) ) {\vert}_{t = {t}_{0}}$$

refers to the estimated ghosting artifact of the time of interest t₀ over the four timeframes output by the neural network.

Once the background signal is estimated according to Eqn. (4), the next step for outer volume removal is to subtract the background signal from the raw k-space data without interfering with the signal around the heart (or other moving object(s)). To this end, an additional neural network may be trained to predict heart boundaries from coil-combined composite images. This was used to generate a mask, m_{OVR}, that outlines the outer volume that needs to be removed, which was specified as everything outside the heart boundaries that were defined using a rectangle in the phase-encode direction spanning the full frequency-encode, since no undersampling is performed in the latter, as illustrated in FIG. 2D. Subsequently, the outer volume removed k-space was generated from the acquired data y^{Ω}^{ₜ} as:

$${y}_{OVR}^{{Ω}_{t}} = {y}^{{Ω}_{t}} - {F}^{{Ω}_{t}} \{ {m}_{OVR} \cdot {x}_{background} \} ; \qquad ( 5 )$$

where F^{Ω}^{ₜ} denotes the Fourier transform operator undersampled at Ωₜ k-space locations.

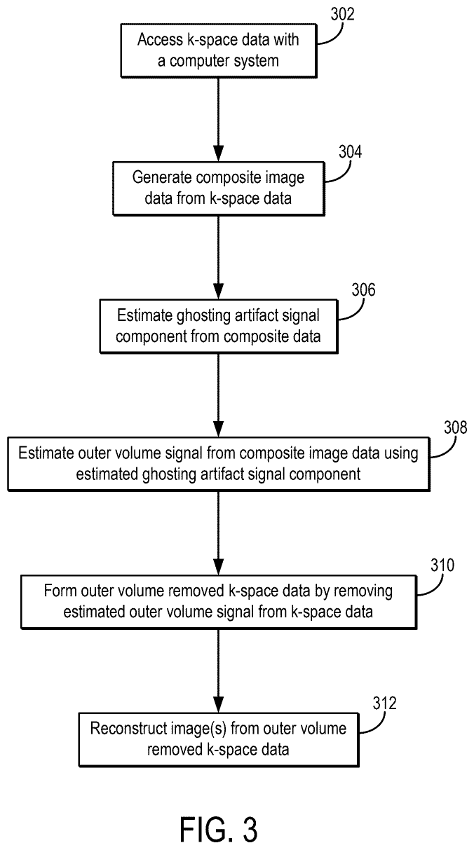

Referring now to FIG. 3, a flowchart is illustrated as setting forth the steps of an example method for reconstructing one or more images from k-space data using a deep learning-based outer volume signal suppression.

The method includes accessing k-space data with a computer system, as indicated at step 302. Accessing the k-space data may include retrieving such data from a memory or other suitable data storage device or medium. Additionally or alternatively, accessing the k-space data may include acquiring such data with an MRI system and transferring or otherwise communicating the data to the computer system, which may be a part of the MRI system.

In general, the k-space data are acquired from a spatial region in a subject that contains a region-of-interest (ROI). The ROI may be an ROI containing an anatomical target of interest, such as the heart, the prostate, the brain, or another organ or portion of the subject's anatomy. The ROI corresponds to a spatial region that is smaller than the spatial region from which the k-space data are acquired (e.g., the imaging field-of-view). The k-space data may be acquired during a time when there is motion in the ROI (e.g., points in the cardiac cycle when the heart is beating), or during a time where there is no motion in the ROI, or at least substantially no motion of the anatomical target in the ROI (e.g., points in the cardiac cycle when the heart is not beating).

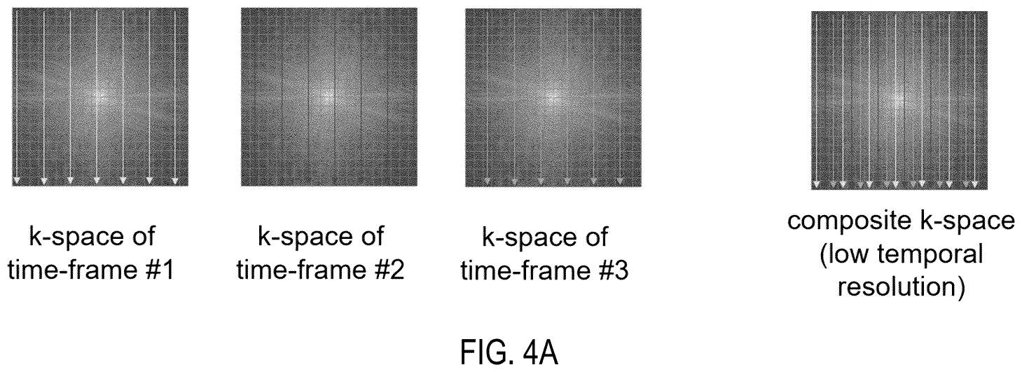

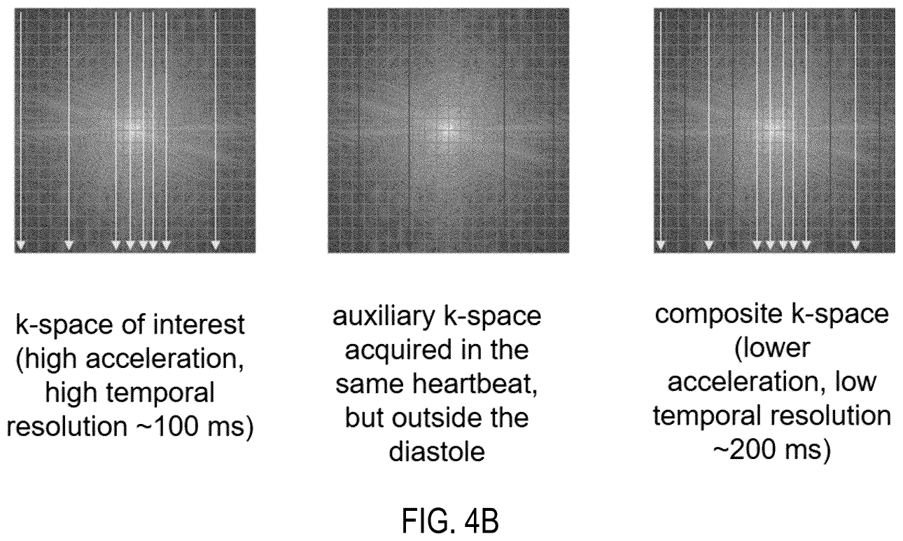

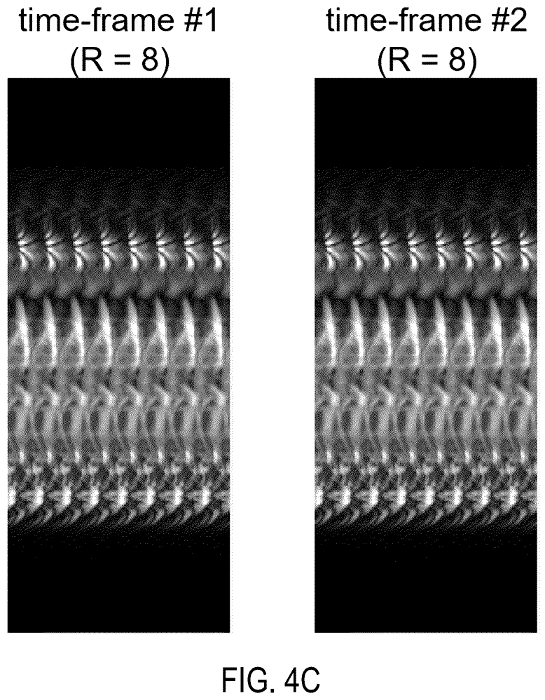

In some embodiments, the k-space data are acquired using a time-interleaved shifted undersampling pattern. An example grouping of time-interleaved undersampling patterns for k-space is illustrated in FIG. 4A. In this example, the multiple frames of k-space data (e.g., three for R=3) are acquired. A low temporal resolution composite image can be generated from the composite k-space data, which has low temporal resolution. Another example of time-interleaved undersampling patterns for k-space is illustrated in FIG. 4B. In this latter example, the k-space of interest (high acceleration, high temporal resolution ˜100 ms) is acquired with a first set of k-space lines and auxiliary k-space data is acquired in the same heartbeat, but outside diastole, using a second set of k-space lines. The composite image in this example has lower acceleration and low temporal resolution, such as ˜200 ms. Advantageously, when acquiring k-space data using a sample pattern such as the one in FIG. 4B where a central portion of k-space is sampled more densely than the peripheral regions of k-space, ghosting artifacts may not need to be explicitly removed from the k-space data (e.g., when the center of k-space is fully sampled with little to no motion). In such instances, the outer volume signals may instead be estimated based on the pulse sequence used for the data acquisition. For example, the pulse sequence used to acquire data can include using the “dead time” in the sequence to acquire additional motion-corrupted data. Normally no data is acquired in these times, since there is too much cardiac motion, but here the additional data can be used to help estimate the outer volume signals. An example of timeframes of k-space data acquired using a time-interleaved undersampling pattern is shown in FIG. 4C.

As an advantage, by acquiring k-space data using time-interleaved shifted undersampling pattern, a fully-sampled composite k-space data set and/or image with low temporal resolution can be formed by merging R adjacent timeframes, where R is the undersampling rate. In still other embodiments, R adjacent timeframes may not be needed (e.g., when using k-space sample patterns such as those in FIG. 4B). For example, as illustrated in FIG. 4B, k-space data were acquired with R=6 and two timeframes. In this example, the overall rate of low-resolution data is R=3, which is still reconstructable. It is an aspect of the present disclosure that this composite image data (i.e., k-space data and/or image) contains both the outer volume signal and a temporally averaged ROI image. When the target anatomy is undergoing motion (e.g., the heart undergoing cardiac motion, another organ experiencing respiratory motion), well as ghosting artifacts resulting from the motion may be superimposed onto the composite image.

Composite image data are, therefore, generated from the k-space data, as indicated at step 304. In a non-limiting example, the composite data can be formed by first decomposing the true image at each timeframe as the sum of the ROI and background. Thus, the contribution of each timeframe to the composite image is R-folded ROI and background image. Additionally, because of the shifting sampling pattern in k-space, foldovers in different timeframes have distinct modulation constants, as illustrated in FIG. 1. In this illustrated example, a dynamic imaging acquisition is used, such as one using the k-space sampling patterns shown in FIG. 4A.

In particular, the ROI foldovers at the true ROI location have no phase and result in a temporally averaged ROI when summed across R timeframes. However, the other ROI foldovers create a pseudo-periodic ghosting artifact in the background due to ROI motion between timeframes. Conversely, the foldovers for the stationary background sum up constructively at the central location, while canceling out for all other ones. Thus, as described above, the composite image x_{composite}(t) at time t can be written as:

$${x}_{c o m} ( t ) = {\overline{x}}_{m o v i n g} ( t ) + {x}_{ghost} ( t ) + {x}_{b a c k g r o u n d} ( t ) ; \qquad ( 6 )$$

where x_{moving}(t) is the temporally-averaged moving components, which may be the moving components in the ROI; x_{ghost}(t) is the ghosting artifact; and x_{background}(t) is the stationary background components that form the basis of the outer volume image.

Alternatively, an imaging acquisition using the k-space sampling patterns shown in FIG. 4B can be used. In these instances, the composite data can be generated as a combination of k-space data acquired in a first time frame that fully samples (or more densely samples) a central region of k-space and in a second time frame that samples peripheral regions of k-space. The ghosting artifacts in these examples may instead be noise-like ghosting that occurs from cardiac motion between the two time frames.

The composite image can be split into these three parts to estimate the outer volume signal. First, a suitably trained machine learning model is used to estimate the pseudo-periodic ghost artifact image from the composite image data, as indicated at step 306. As a non-limiting example, the step of estimating the pseudo-periodic ghosting artifact component of the composite image can include accessing a suitably trained machine learning model with the computer system. In general, the machine learning model is trained, or has been trained, on training data in order to estimate the pseudo-periodic ghosting artifact component of the composite image described above.

Accessing the machine learning model may include accessing model parameters (e.g., weights, biases, or both) that have been optimized or otherwise estimated by training the machine learning model on training data. In some instances, retrieving the machine learning model can also include retrieving, constructing, or otherwise accessing the particular model architecture to be implemented. For instance, data pertaining to the layers in a neural network architecture (e.g., number of layers, type of layers, ordering of layers, connections between layers, hyperparameters for layers) may be retrieved, selected, constructed, or otherwise accessed.

An artificial neural network generally includes an input layer, one or more hidden layers (or nodes), and an output layer. Typically, the input layer includes as many nodes as inputs provided to the artificial neural network. The number (and the type) of inputs provided to the artificial neural network may vary based on the particular task for the artificial neural network.

The input layer connects to one or more hidden layers. The number of hidden layers varies and may depend on the particular task for the artificial neural network. Additionally, each hidden layer may have a different number of nodes and may be connected to the next layer differently. For example, each node of the input layer may be connected to each node of the first hidden layer. The connection between each node of the input layer and each node of the first hidden layer may be assigned a weight parameter. Additionally, each node of the neural network may also be assigned a bias value. In some configurations, each node of the first hidden layer may not be connected to each node of the second hidden layer. That is, there may be some nodes of the first hidden layer that are not connected to all of the nodes of the second hidden layer. The connections between the nodes of the first hidden layers and the second hidden layers are each assigned different weight parameters. Each node of the hidden layer is generally associated with an activation function. The activation function defines how the hidden layer is to process the input received from the input layer or from a previous input or hidden layer. These activation functions may vary and be based on the type of task associated with the artificial neural network and also on the specific type of hidden layer implemented.

Each hidden layer may perform a different function. For example, some hidden layers can be convolutional hidden layers which can, in some instances, reduce the dimensionality of the inputs. Other hidden layers can perform statistical functions such as max pooling, which may reduce a group of inputs to the maximum value; an averaging layer; batch normalization; and other such functions. In some of the hidden layers each node is connected to each node of the next hidden layer, which may be referred to then as dense layers. Some neural networks including more than, for example, three hidden layers may be considered deep neural networks.

The last hidden layer in the artificial neural network is connected to the output layer.

Let x_{composite}(t₀) denote the composite image centered around a timeframe of interest, t₀. As model input, a concatenation of a number of such composite images can be used to cover a wider temporal window. For example, a concatenation of four composite images, such as

$${\{ {x}_{c o mposite}^{concat} ( τ ) \}}_{τ = {t}_{0} - 1}^{{t}_{0} + 2} ,$$

can be used. The output of the machine learning model is defined as the ghosting component of the composite image for timeframe t₀.

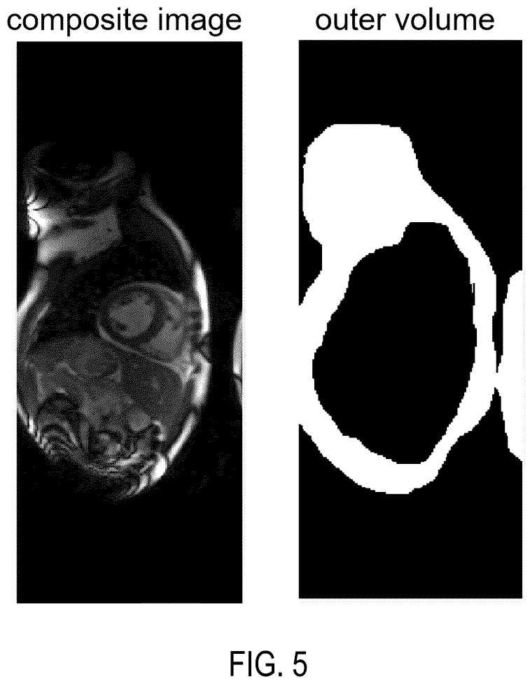

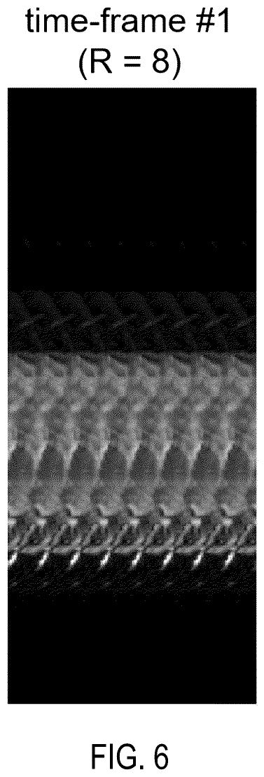

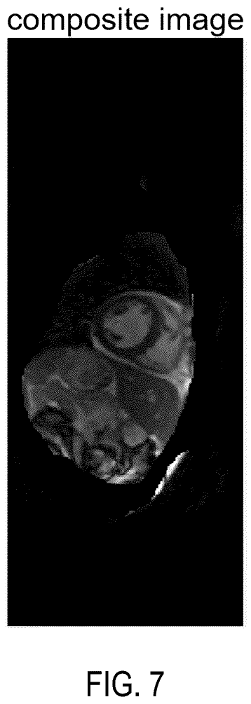

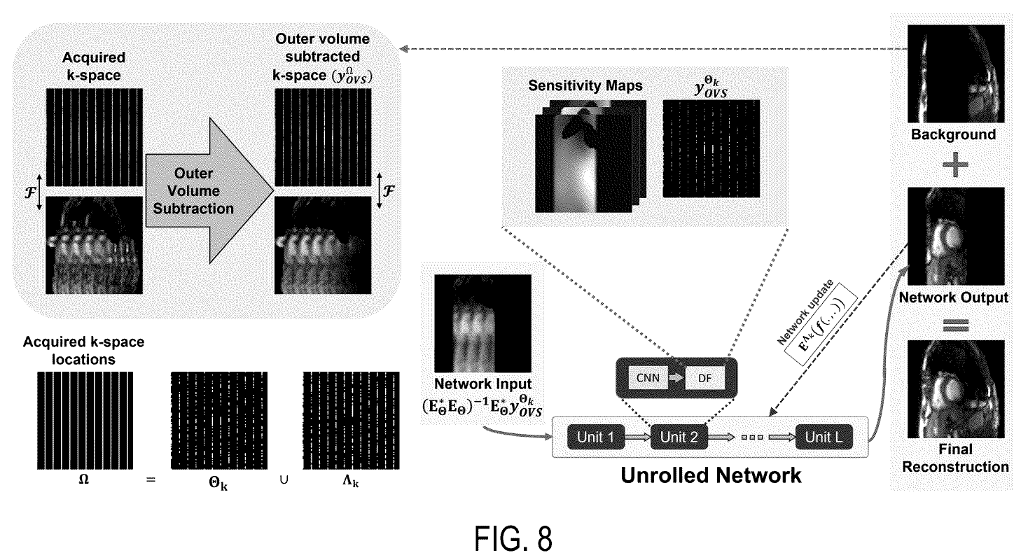

The outer volume signal in the k-space data is then estimated, as indicated at step 308. In general, the background, outer volume signal can be estimated by subtracting the estimated ghosting artifact component generated by the machine learning model from the composite image. An example of a composite mage and an image mask corresponding to the estimated outer volume signals from the composite image is illustrated in FIG. 5. Outer volume removed k-space data are then generated by removing the estimated outer volume signal from the acquired k-space data for each timeframe, as indicated at step 310. As a result, sub-sampled k-space corresponding to an image with no signal outside of the ROI are obtained. An example of a timeframe of outer volume removed k-space data is illustrated in FIG. 6. The outer volume signal data may also be subtracted from the composite image to generate outer volume removed composite image, which can be used as outer volume removed calibration data for image reconstruction. An example of an outer volume removed composite image is shown in FIG. 7.

One or more images of the subject are then reconstructed from the outer volume removed k-space data, as indicated at step 312. As one non-limiting example, the image(s) may be reconstructed from the outer volume removed k-space data using a physics-driven deep learning model. Advantageously, by removing the outer volume signal from the k-space data, the reconstruction problem is made easier for solving with a physics-driven deep learning model.

As an example, let

$${y}_{OVR}^{{Ω}_{t}}$$

be the outer volume removed k-space signal with Ωₜ as the undersampling pattern used when acquiring the original k-space data for timeframe t. In general, a physics-driven deep learning reconstruction uses a regularized least squares objective to reconstruct an image, such as the following:

$$arg {min}_{{x}_{O V R}} {‖ {y}_{OVR}^{{Ω}_{t}} - {E}^{{Ω}_{t}} {x}_{O V R} ‖}_{2}^{2} + R ( {x}_{O V R} ) ; \qquad ( 7 )$$

where E^{Ω}^{ₜ} is a multi-coil encoding operator with a sub-sampled Fourier operator that samples Ωₜ and coil sensitivity maps (e.g., outer volume masked out sensitivities such as masked coil sensitivity maps), and x_{OVR} represents the target image with the outer volume removed. The first term in the objective promotes data fidelity (DF) that ensures consistency with the acquired k-space data, while R(⋅) is a regularization term that imposes prior constraints on the reconstruction. One technique for implementing a physics-driven deep learning reconstruction uses an unrolled iterative algorithm for solving Eqn. (7) for a fixed number of iterations, where the proximal operator corresponding to R(⋅) can be implicitly modeled using a neural network, while the DF term weights may be learned during end-to-end training. An example unrolled network structure is illustrated in FIGS. 2B and 2C and its implementation and example final image formation is illustrated in FIGS. 2D and 2E.

For highly-accelerated rates, a self-supervised learning approach (SSDU) may be used for training without fully-sampled reference data. As example of an SSDU based approach is described in co-pending U.S. patent application Ser. No. 17/075,411, which is herein incorporated by reference in its entirety. As an example, multi-mask SSDU splits the acquired k-space data locations, Ω, into pairs of two disjoint sets: {Θₖ, Λₖ} where the first set, Θₖ, is used in the DF units of the unrolled network during training, while the second set, Λₖ, is used to define the self-supervised training loss, leading to the loss function:

$${min}_{γ} 𝔼 [ \frac{1}{K} {\sum }_{k = 1}^{K} ℒ ( {y}_{OVR}^{{Λ}_{t}^{k}} , {E}^{{Λ}_{t}^{k}} ( {g}_{γ} ( {y}_{OVR}^{{Θ}_{t}^{k}} , {E}^{{Θ}_{t}^{k}} ) ) ) ] ; \qquad ( 8 )$$

where

$${g}_{γ} ( {y}_{OVR}^{{Θ}_{t}^{k}} , {E}^{{Θ}_{t}^{k}} )$$

is the output of the physics-driven deep learning network with learnable parameters, γ, for input measurements

$${y}_{OVR}^{{Θ}_{t}^{k}}$$

and encoding operator

$${E}^{{Θ}_{t}^{k}} ;$$

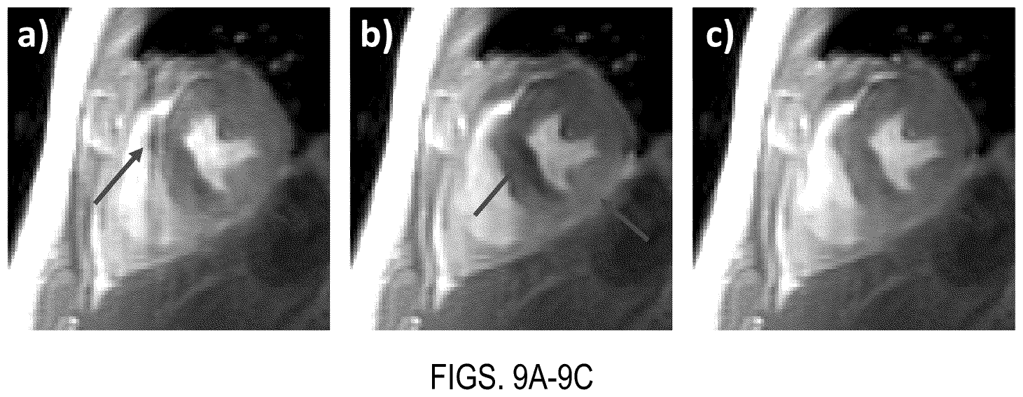

K is the number of SSDU masks; and [symbol] (⋅,⋅⋅) is a loss function, such as a normalized [symbol] ₁- [symbol] ₂ loss function. The training pipeline for reconstruction using this physics-driven deep learning model is summarized in FIG. 9.

While this loss may be used, in some cases additional considerations may be used for outer volume removal. If the outer volume is substantially removed from

$${y}_{OVR}^{{Λ}_{t}^{k}} ,$$

then the corresponding pixels in x_{OVR} are zero. Note that this is a stronger condition than sparsity in compressed sensing, since the location of the zero coefficients are specified as well. This can be enforced either via masking in image domain, or in the context of PD-DL, through setting the coil sensitivities in the outer volume region to zero in

$${E}^{{Λ}_{t}^{k}}$$

in order to not to affect the learning of the proximal operator. The latter can also be viewed as inverting a lower rank system in the context of SENSE. These sensitivity maps may be referred to as “masked” sensitivity maps, whereas the unaltered maps may be referred to as “full” sensitivity maps.

This represents an idealized scenario, as some residual outer volume signal will remain in k-space. If masked sensitivity maps are used, any residual signal may be mapped into the retained pixels of x_{OVR}, potentially introducing artifacts into the ROI, as illustrated in FIG. 9A. Conversely, using full sensitivity maps results in a more challenging reconstruction problem and carries the risk of redistributing the ROI signal into the outer volume, leading to potential signal loss within the ROI, as illustrated in FIG. 9B.

Thus, it can be advantageous to combine the strengths of both approaches. In these cases, a different loss function for training a PD-DL network for outer volume removal may be used. Given the inherent risk of residual outer volume signal within the ROI, which cannot be reversed, the use of full sensitivity maps is advantageous for reconstruction. Nonetheless, the masked sensitivity maps may be used in an additional term to ensure consistency in the ROI signal. In particular, a PD-DL network can first be trained for outer volume removed k-space data using masked sensitivity maps with the loss function in Eqn. (8). Let

$${x}_{O V R}^{m a s k e d}$$

denote the reconstructed images obtained from this network. To avoid issues related to signal loss by naively using the full sensitivity maps, a loss function term that encourages consistency between reconstructions obtained with masked and full sensitivity maps within the ROI can be introduced:

$${min}_{γ} [ \frac{1}{K} {\sum }_{k = 1}^{K} ℒ ( {y}_{OVR}^{{Λ}_{t}^{k}} , {E}^{{Λ}_{t}^{k}} ( {g}_{γ} ( {y}_{OVR}^{{Θ}_{t}^{k}} , {E}^{{Θ}_{t}^{k}} ) ) ) ] + λℒ ( {x}_{O V R}^{m a s k e d} , {g}_{γ} ( {y}_{OVR}^{{Ω}_{t}} , {E}^{{Ω}_{t}} ) \cdot {m}_{OVR} ) ; \qquad ( 9 )$$

where λ is a weight term, and the multi-coil operator E^{Θ}^{ₜ} is defined with full sensitivity maps. This loss function penalizes discrepancies between the two reconstructions, guiding the network to preserve the signal within the ROI, while allowing any residual outer volume signal to be mapped outside the ROI, thereby suppressing artifacts. As shown in FIG. 9C, the updated network effectively eliminates both unwanted artifacts and signal loss in the ROI.

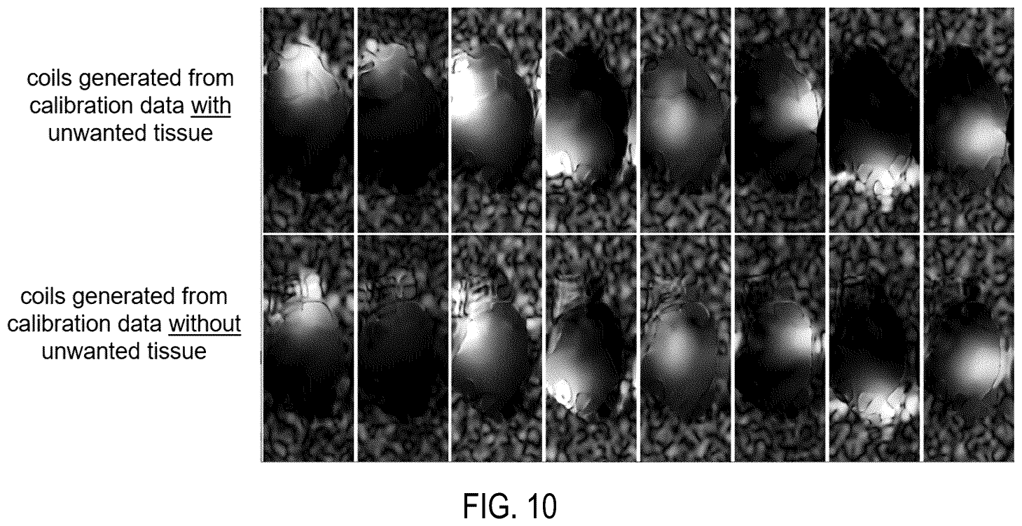

The zero-filled images given to the network generated from the outer volume removed k-space data and masked sensitivity maps can be used as the input to the physics-driven deep learning network. Thus, in some embodiments, masked sensitivity maps are generated or otherwise accessed using the computer system and input to the reconstruction model together with the outer volume removed k-space data. As noted above, the masked coil sensitivity maps can be generated by applying a mask that masks out the outer volume regions in the coil sensitivity maps. Such masks may be generated using the estimated outer volume signals to delineate the spatial region(s) containing outer volume signals. An example of coil sensitivity maps generated from calibration data without outer volume removal (i.e., with unwanted tissue) and coil sensitivity maps generated from calibration data with outer volume removal (i.e., without unwanted tissue) is shown in FIG. 10.

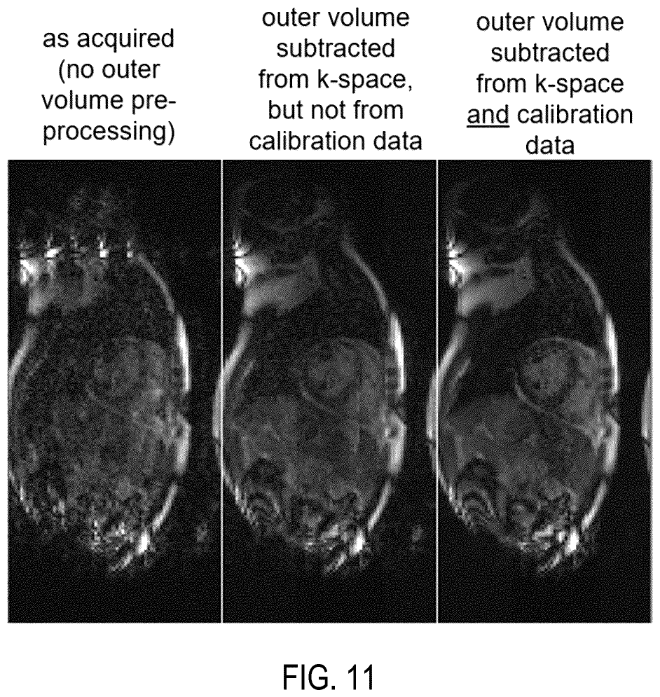

A comparative example of images reconstructed with no outer volume removal, with outer volume removal and using regular calibration data, and with outer volume removal and using outer volume removed calibration data is shown in FIG. 11.

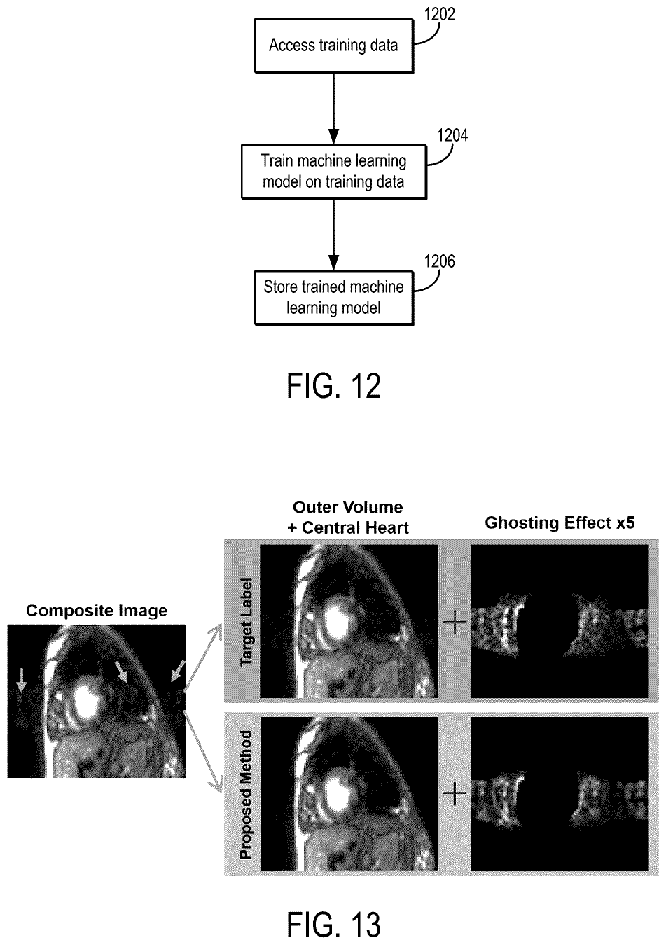

Referring now to FIG. 12, a flowchart is illustrated as setting forth the steps of an example method for training a machine learning model (e.g., a neural network or other deep learning model) on training data, such that the machine learning model is trained to receive a composite image as input data in order to generate ghosting artifact signal component data as an output. In general, the machine learning model can implement any number of different machine learning model architectures. For instance, the machine learning model could implement a convolutional neural network, a residual neural network, other deep learning networks, or the like. Alternatively, the machine learning model could be replaced with other suitable machine learning or artificial intelligence models, such as those based on supervised learning, unsupervised learning, deep learning, ensemble learning, dimensionality reduction, and so on.

The method includes accessing training data with a computer system, as indicated at step 1202. Accessing the training data may include retrieving such data from a memory or other suitable data storage device or medium. Alternatively, accessing the training data may include acquiring such data with an MRI system and transferring or otherwise communicating the data to the computer system.

In general, the training data can include magnetic resonance images and separately labeled ghosting artifact images. In a non-limiting example, training labels can be generated based on baseline parallel image reconstructions (e.g., TGRAPPA reconstructions) performed at a lower accelerated acquisition rate. Subsequently, the ghosting artifact images can be generated at a higher, target acceleration by summing folded and modulated ROI images, cropped from the baseline images using a mask.

The method can therefore include assembling training data from magnetic resonance images using a computer system. This step may include assembling the images into an appropriate data structure on which the machine learning model can be trained. Assembling the training data may include assembling images, segmented images, and other relevant data. For instance, assembling the training data may include generating labeled data and including the labeled data in the training data. Labeled data may include images, segmented images, or other relevant data that have been labeled as belonging to, or otherwise being associated with, one or more different classifications or categories. For instance, labeled data may include images and/or segmented images that have been labeled as being associated with ghosting artifacts (i.e., the ghosting artifact images).

The machine learning model is then trained on the training data, as indicated at step 1204. In general, the machine learning model can be trained by optimizing model parameters (e.g., weights, biases, or both) based on minimizing a loss function. As one non-limiting example, the training can be performed by minimizing the following:

$${min}_{θ} {𝔼}_{{x}_{g h o s t}^{r e f} ( t )} ℒ ( {x}_{ghost}^{r e f} ( t ) , {f}_{θ} ( {x}_{c o m}^{concat} ( t ) ) \cdot M ( t ) ) ; \qquad ( 10 )$$

where f_{θ}(⋅) denotes the network output with parameters θ, and M(⋅) is the mask that covers ghosting artifacts excluding the true ROI location to ensure that the averaged ROI image, which has a much higher intensity than ghosting, does not dominate the loss function. In some implementations, the mask, M(⋅) may be generated by a segmentation artificial neural network.

As a non-limiting example, training a neural network may include initializing the neural network, such as by computing, estimating, or otherwise selecting initial network parameters (e.g., weights, biases, or both). During training, an artificial neural network receives the inputs for a training example and generates an output using the bias for each node, and the connections between each node and the corresponding weights. For instance, training data can be input to the initialized neural network, generating output as ghosting artifact images. The artificial neural network then compares the generated output with the actual output of the training example in order to evaluate the quality of the ghosting artifact images. For instance, the ghosting artifact images can be passed to a loss function to compute an error. The current neural network can then be updated based on the calculated error (e.g., using backpropagation methods based on the calculated error). For instance, the current neural network can be updated by updating the network parameters (e.g., weights, biases, or both) in order to minimize the loss according to the loss function. The training continues until a training condition is met. The training condition may correspond to, for example, a predetermined number of training examples being used, a minimum accuracy threshold being reached during training and validation, a predetermined number of validation iterations being completed, and the like. When the training condition has been met (e.g., by determining whether an error threshold or other stopping criterion has been satisfied), the current neural network and its associated network parameters represent the trained neural network. Different types of training processes can be used to adjust the bias values and the weights of the node connections based on the training examples. The training processes may include, for example, gradient descent, Newton's method, conjugate gradient, quasi-Newton, Levenberg-Marquardt, among others.

The machine learning model is then stored for later use, as indicated at step 1206. Storing the machine learning model may include storing model parameters (e.g., weights, biases, or both), which have been computed or otherwise estimated by training the machine learning model on the training data. Storing the trained machine learning model may also include storing the particular model architecture to be implemented. For instance, data pertaining to the layers in a neural network architecture (e.g., number of layers, type of layers, ordering of layers, connections between layers, hyperparameters for layers) may be stored.

FIG. 13 shows representative results for ghosting artifact detection using the proposed machine learning model approach (bottom row) compared to target labels (top row). Visibly similar pseudo-periodic patterns are observed in the ghosting artifacts, confirming the efficacy of the proposed approach. It is also noted that the target label is noisier as it was generated using TGRAPPA at R=4, and thus contains spatially-varying g-factor noise, whereas the methods described in the present disclosure uses higher-SNR composite images as input. The middle column shows the outer volume+central heart images, generated by subtracting the estimated ghosting artifacts from the composite image. These images indicate that the proposed approach leads to an artifact-free background image, removing the aliasing in the composite images (arrows).

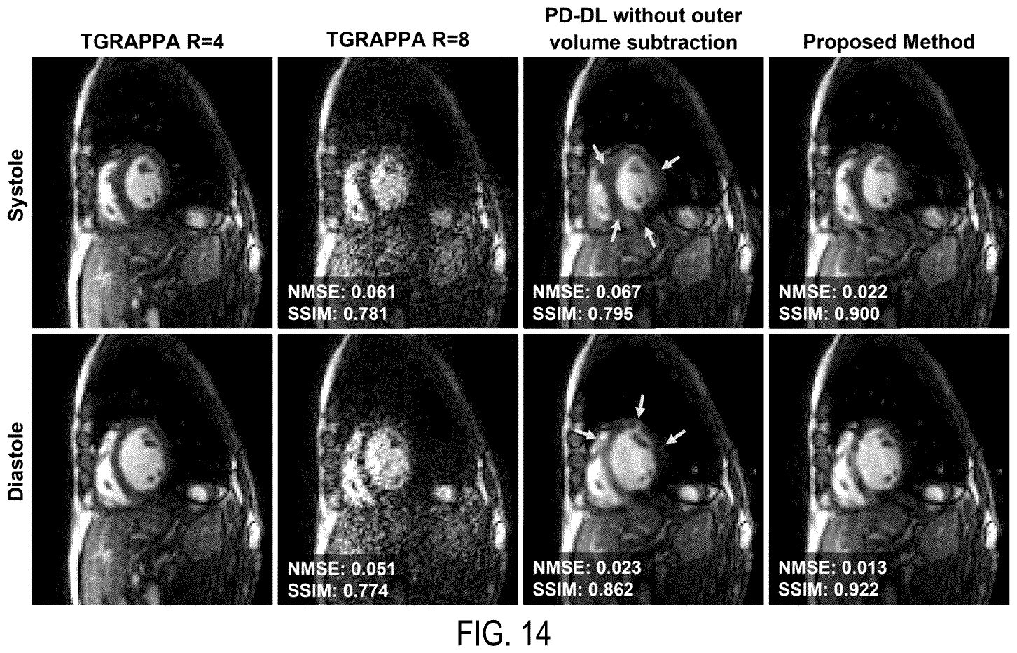

FIG. 14 displays two representative timeframes corresponding to systolic and diastolic cardiac phases for a retrospectively R=8 accelerated real-time cine dataset. The disclosed method (rightmost column) at R=8 visibly improves on both the conventional TGRAPPA at the same rate and the physics-driven deep learning reconstruction without outer volume subtraction, while exhibiting similar image quality to TGRAPPA at baseline R=4 with a clear depiction of myocardium-blood boundaries at R=8. The importance of the proposed outer volume subtraction pipeline is evident as the physics-driven deep learning reconstruction without outer volume subtraction (third column) exhibits significant aliasing and blurring artifacts in myocardium-blood boundaries and papillary muscles, highlighting that physics-driven deep learning alone is insufficient for reconstruction at this high acceleration rate. NMSE and SSIM values are reported with respect to the baseline R=4 reconstruction while noting these are not reference datasets due to the aforementioned g-factor noise amplification.

In an example study, the systems and methods described in the present disclosure were used for outer volume removal in ECG-triggered cardiac MRI sequences used for anatomical imaging. A pulse sequence that capitalizes on the gaps in the R-R interval present in these ECG-triggered sequences was implemented. Specifically, auxiliary data were acquired after a primary imaging window, and these auxiliary data are used to estimate the outer volume. These outer volume signals are then subtracted from the imaging window data, thereby maintaining the high spatiotemporal resolution of the imaging signal. Additionally, an analytical characterization of ghosting and aliasing artifacts associated with the acquisition method was performed, examining how outer volume signals can be effectively removed. The outer volume removal approach simplified the reconstruction process, which was solved using a DL-based approach to reconstruct a background-free, artifact-reduced imaging signal. This strategy enabled clearer, high-resolution cardiac imaging with minimized motion artifacts and improved robustness for high-acceleration cardiac MRI.

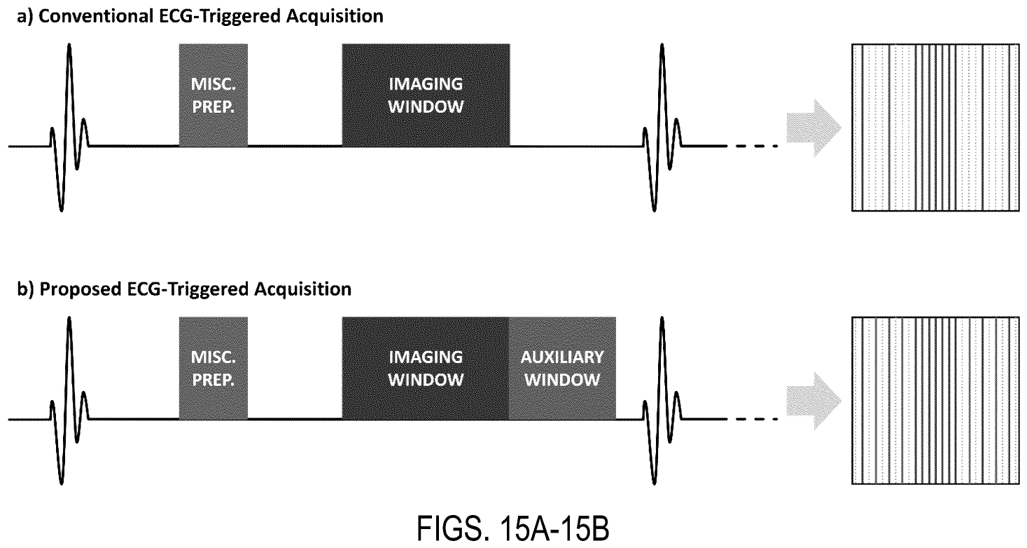

Conventional ECG-triggered sequences acquire data after preparation pulses (e.g., T2-prep or inversion) with an acceleration rate of R. A representative single-shot sequence incorporating auto-calibration signal (ACS) lines is illustrated with red lines in FIG. 15A. The ECG-triggered pulse sequence used in the example study continued acquisition by filling in the intermediate signals between the acquired lines as auxiliary lines immediately following the imaging window (green lines in FIG. 15B). Even though these auxiliary lines are corrupted in terms of cardiac motion, the outer volume signal is expected to be stationary during this window. As a result, the outer volume signal can be estimated from an acquisition with lower effective acceleration rate.

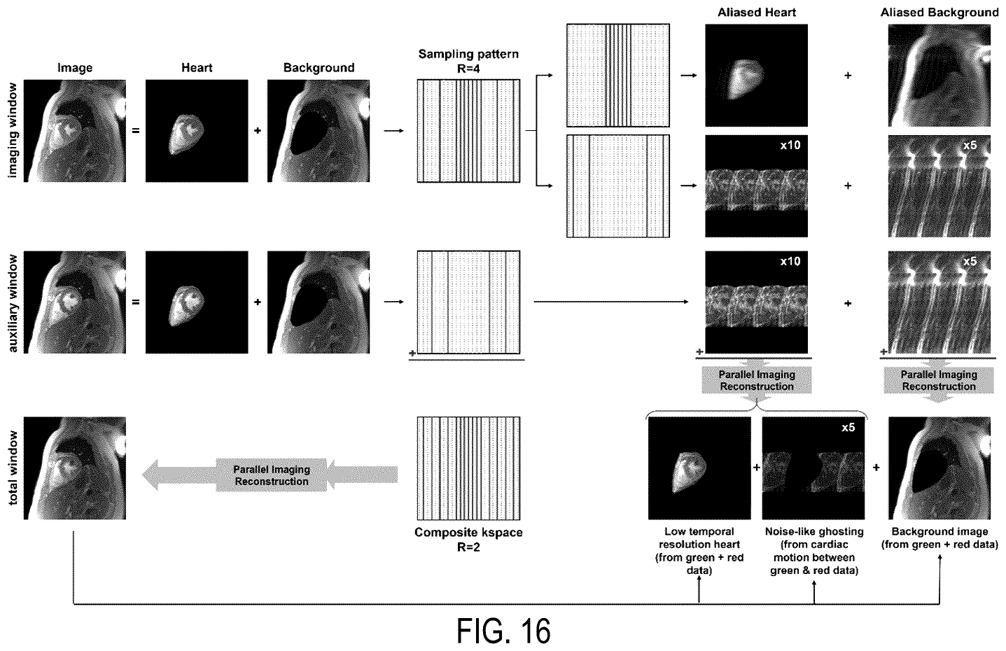

For outer volume signal removal, the systems and methods described in the present disclosure were implemented. All acquired k-space data were used to estimate the background signal after reconstruction (FIG. 16). The low-frequency components of the total signal were captured during the imaging window, while ghosting artifacts from cardiac motion in the auxiliary data only affect the high-frequency components in k-space, and correspondingly edge-like structures in image domain. As a result, these ghosting artifacts appear as noise-like interference, which can be removed with DL reconstruction without requiring an additional explicit ghosting detection step as in steady-state sequences. The outer volume signal was subsampled with the imaging window sampling pattern and removed from the imaging signal, yielding a background-free signal that retains the same spatiotemporal resolution as the original imaging window.

The imaging sequence was tested on 2D real-time LGE CMR acquired in 1 heartbeat/slice. LGE data were obtained at 1.5 T as multi-slice acquisition with 15 slices using our sequence at R=2 with 15 ACS lines, resolution=1.5×1.5 mm², FOV=300×300 mm², slice thickness=5 mm. After outer volume removal, a slice-specific physics-driven DL reconstruction network was trained with ZS-SSDU using K=25 masks over 100 epochs by unrolling a physics-driven deep-learning (PD-DL) network for 10 times. The proximal operator for the regularizer was implemented with a ResNet with 15 residual blocks.

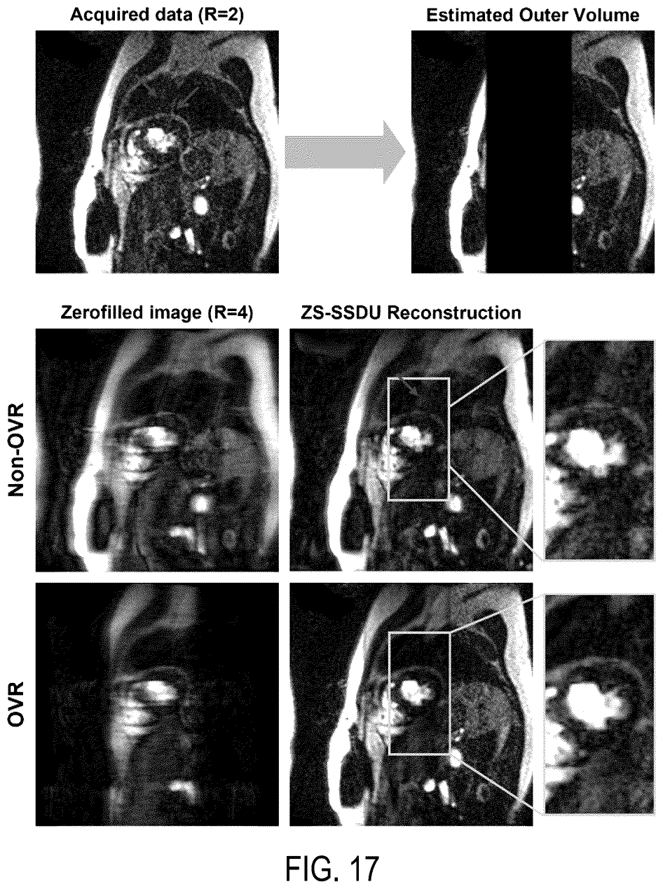

FIG. 17 depicts representative reconstruction results, including the estimated outer volume, and ZS-SSDU reconstructions at R=4 with and without outer volume removal. The disclosed pipeline enabled clear depiction of myocardium-blood boundaries by clearing the artifacts coming from the outer volume.

In this example study, the outer volume removal techniques described in the present disclosure were used for fast, ECG-triggered anatomical cardiac MRI. A pulse sequence that leveraged the gap in the R-R interval was used to collect auxiliary signals. Using all acquired data, outer volume estimation was performed. The estimated outer volume was then subtracted from the imaging window signal, thus preserving the spatiotemporal resolution of the acquisition, and these were reconstructed using ZS-SSDU. The feasibility results at a nominal acceleration rate of R=4 show that the disclosed pipeline demonstrates superior image quality compared to the conventional approach without outer volume removal for real-time LGE. Further gains may be possible at higher acceleration rates and for 3D sequences.

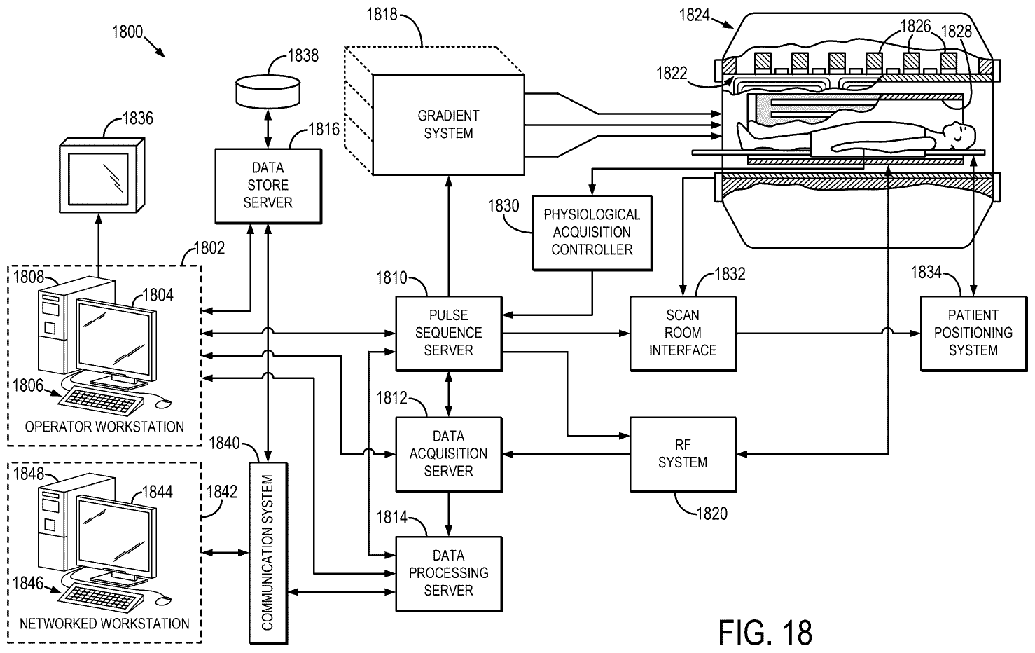

Referring particularly now to FIG. 18, an example of an MRI system 1800 that can implement the methods described here is illustrated. The MRI system 1800 includes an operator workstation 1802 that may include a display 1804, one or more input devices 1806 (e.g., a keyboard, a mouse), and a processor 1808. The processor 1808 may include a commercially available programmable machine running a commercially available operating system. The operator workstation 1802 provides an operator interface that facilitates entering scan parameters into the MRI system 1800. The operator workstation 1802 may be coupled to different servers, including, for example, a pulse sequence server 1810, a data acquisition server 1812, a data processing server 1814, and a data store server 1816. The operator workstation 1802 and the servers 1810, 1812, 1814, and 1816 may be connected via a communication system 1840, which may include wired or wireless network connections.

The pulse sequence server 1810 functions in response to instructions provided by the operator workstation 1802 to operate a gradient system 1818 and a radiofrequency (“RF”) system 1820. Gradient waveforms for performing a prescribed scan are produced and applied to the gradient system 1818, which then excites gradient coils in an assembly 1822 to produce the magnetic field gradients Gₓ, G_{y}, and G_{z} that are used for spatially encoding magnetic resonance signals. The gradient coil assembly 1822 forms part of a magnet assembly 1824 that includes a polarizing magnet 1826 and a whole-body RF coil 1828.

RF waveforms are applied by the RF system 1820 to the RF coil 1828, or a separate local coil to perform the prescribed magnetic resonance pulse sequence. Responsive magnetic resonance signals detected by the RF coil 1828, or a separate local coil, are received by the RF system 1820. The responsive magnetic resonance signals may be amplified, demodulated, filtered, and digitized under direction of commands produced by the pulse sequence server 1810. The RF system 1820 includes an RF transmitter for producing a wide variety of RF pulses used in MRI pulse sequences. The RF transmitter is responsive to the prescribed scan and direction from the pulse sequence server 1810 to produce RF pulses of the desired frequency, phase, and pulse amplitude waveform. The generated RF pulses may be applied to the whole-body RF coil 1828 or to one or more local coils or coil arrays.

The RF system 1820 also includes one or more RF receiver channels. An RF receiver channel includes an RF preamplifier that amplifies the magnetic resonance signal received by the coil 1828 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

$$M = \sqrt{{I}^{2} + {Q}^{2}} ;$$

and the phase of the received magnetic resonance signal may also be determined according to the following relationship:

$$φ = {tan}^{- 1} ( \frac{Q}{I} ) .$$

The pulse sequence server 1810 may receive patient data from a physiological acquisition controller 1830. By way of example, the physiological acquisition controller 1830 may receive signals from a number of different sensors connected to the patient, including electrocardiograph (“ECG”) signals from electrodes, or respiratory signals from a respiratory bellows or other respiratory monitoring devices. These signals may be used by the pulse sequence server 1810 to synchronize, or “gate,” the performance of the scan with the subject's heart beat or respiration.

The pulse sequence server 1810 may also connect to a scan room interface circuit 1832 that receives signals from various sensors associated with the condition of the patient and the magnet system. Through the scan room interface circuit 1832, a patient positioning system 1834 can receive commands to move the patient to desired positions during the scan.

The digitized magnetic resonance signal samples produced by the RF system 1820 are received by the data acquisition server 1812. The data acquisition server 1812 operates in response to instructions downloaded from the operator workstation 1802 to receive the real-time magnetic resonance data and provide buffer storage, so that data is not lost by data overrun. In some scans, the data acquisition server 1812 passes the acquired magnetic resonance data to the data processor server 1814. In scans that require information derived from acquired magnetic resonance data to control the further performance of the scan, the data acquisition server 1812 may be programmed to produce such information and convey it to the pulse sequence server 1810. For example, during pre-scans, magnetic resonance data may be acquired and used to calibrate the pulse sequence performed by the pulse sequence server 1810. As another example, navigator signals may be acquired and used to adjust the operating parameters of the RF system 1820 or the gradient system 1818, or to control the view order in which k-space is sampled. In still another example, the data acquisition server 1812 may also process magnetic resonance signals used to detect the arrival of a contrast agent in a magnetic resonance angiography (“MRA”) scan. For example, the data acquisition server 1812 may acquire magnetic resonance data and processes it in real-time to produce information that is used to control the scan.

The data processing server 1814 receives magnetic resonance data from the data acquisition server 1812 and processes the magnetic resonance data in accordance with instructions provided by the operator workstation 1802. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 1814 are conveyed back to the operator workstation 1802 for storage. Real-time images may be stored in a data base memory cache, from which they may be output to operator display 1802 or a display 1836. Batch mode images or selected real time images may be stored in a host database on disc storage 1838. When such images have been reconstructed and transferred to storage, the data processing server 1814 may notify the data store server 1816 on the operator workstation 1802. The operator workstation 1802 may be used by an operator to archive the images, produce films, or send the images via a network to other facilities.

The MRI system 1800 may also include one or more networked workstations 1842. For example, a networked workstation 1842 may include a display 1844, one or more input devices 1846 (e.g., a keyboard, a mouse), and a processor 1848. The networked workstation 1842 may be located within the same facility as the operator workstation 1802, or in a different facility, such as a different healthcare institution or clinic.

The networked workstation 1842 may gain remote access to the data processing server 1814 or data store server 1816 via the communication system 1840. Accordingly, multiple networked workstations 1842 may have access to the data processing server 1814 and the data store server 1816. In this manner, magnetic resonance data, reconstructed images, or other data may be exchanged between the data processing server 1814 or the data store server 1816 and the networked workstations 1842, such that the data or images may be remotely processed by a networked workstation 1842.

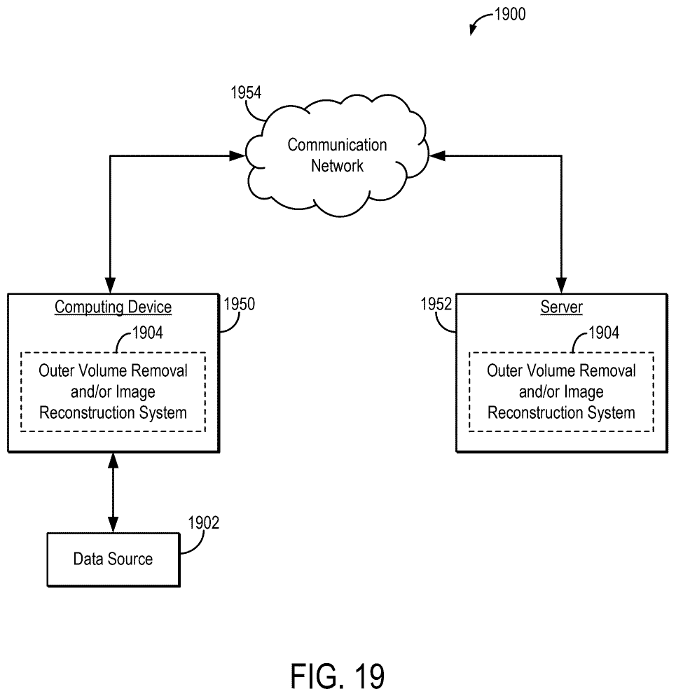

FIG. 19 shows an example of a system 1900 for reconstructing images with outer volume removal in accordance with some embodiments described in the present disclosure. As shown in FIG. 19, a computing device 1950 can receive one or more types of data (e.g., k-space data) from data source 1902. In some embodiments, computing device 1950 can execute at least a portion of an outer volume signal removal and/or image reconstruction system 1904 to suppress outer volume signals and/or reconstruct images from data received from the data source 1902.

Additionally or alternatively, in some embodiments, the computing device 1950 can communicate information about data received from the data source 1902 to a server 1952 over a communication network 1954, which can execute at least a portion of the outer volume signal removal and/or image reconstruction system 1904. In such embodiments, the server 1952 can return information to the computing device 1950 (and/or any other suitable computing device) indicative of an output of the outer volume signal removal and/or image reconstruction system 1904.

In some embodiments, computing device 1950 and/or server 1952 can be any suitable computing device or combination of devices, such as a desktop computer, a laptop computer, a smartphone, a tablet computer, a wearable computer, a server computer, a virtual machine being executed by a physical computing device, and so on. The computing device 1950 and/or server 1952 can also reconstruct images from the data.

In some embodiments, data source 1902 can be any suitable source of data (e.g., k-space data, images reconstructed from k-space data, processed k-space data, processed images), such as an MRI system, another computing device (e.g., a server k-space data, images reconstructed from k-space data, processed k-space data, processed images), and so on. In some embodiments, data source 1902 can be local to computing device 1950. For example, data source 1902 can be incorporated with computing device 1950 (e.g., computing device 1950 can be configured as part of a device for measuring, recording, estimating, acquiring, or otherwise collecting or storing data). As another example, data source 1902 can be connected to computing device 1950 by a cable, a direct wireless link, and so on. Additionally or alternatively, in some embodiments, data source 1902 can be located locally and/or remotely from computing device 1950, and can communicate data to computing device 1950 (and/or server 1952) via a communication network (e.g., communication network 1954).

In some embodiments, communication network 1954 can be any suitable communication network or combination of communication networks. For example, communication network 1954 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 1954 can be a local area network, a wide area network, a public network (e.g., the Internet), a private or semi-private network (e.g., a corporate or university intranet), any other suitable type of network, or any suitable combination of networks. Communications links shown in FIG. 19 can each be any suitable communications link or combination of communications links, such as wired links, fiber optic links, Wi-Fi links, Bluetooth links, cellular links, and so on.

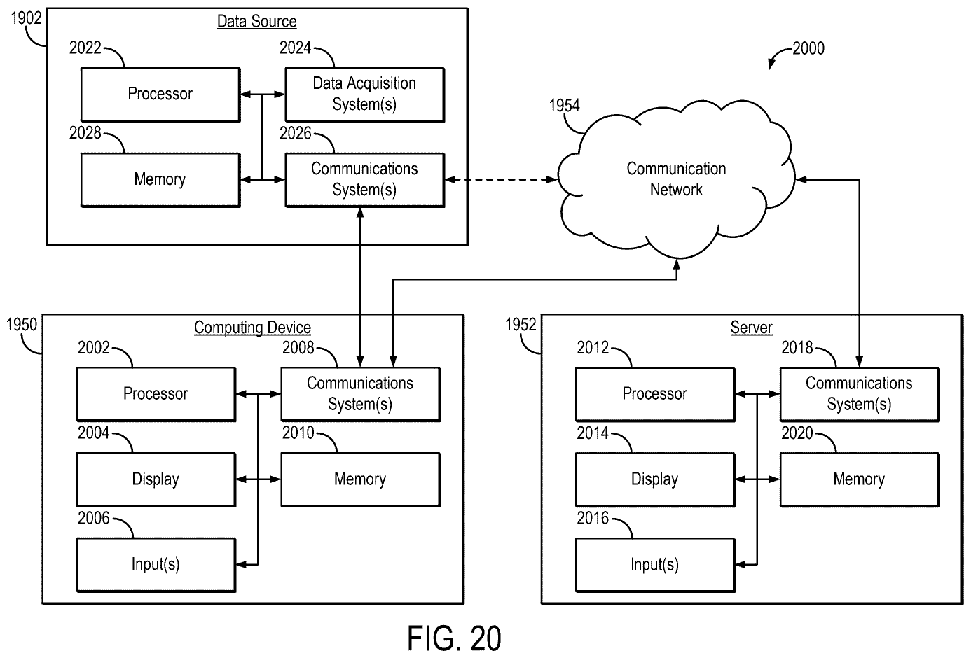

Referring now to FIG. 20, an example of hardware 2000 that can be used to implement data source 1902, computing device 1950, and server 1952 in accordance with some embodiments of the systems and methods described in the present disclosure is shown.

As shown in FIG. 20, in some embodiments, computing device 1950 can include a processor 2002, a display 2004, one or more inputs 2006, one or more communication systems 2008, and/or memory 2010. In some embodiments, processor 2002 can be any suitable hardware processor or combination of processors, such as a central processing unit (CPU), a graphics processing unit (GPU), and so on. In some embodiments, display 2004 can include any suitable display devices, such as a liquid crystal display (LCD) screen, a light-emitting diode (LED) display, an organic LED (OLED) display, an electrophoretic display (e.g., an “e-ink” display), a computer monitor, a touchscreen, a television, and so on. In some embodiments, inputs 2006 can include any suitable input devices and/or sensors that can be used to receive user input, such as a keyboard, a mouse, a touchscreen, a microphone, and so on.

In some embodiments, communications systems 2008 can include any suitable hardware, firmware, and/or software for communicating information over communication network 1954 and/or any other suitable communication networks. For example, communications systems 2008 can include one or more transceivers, one or more communication chips and/or chip sets, and so on. In a more particular example, communications systems 2008 can include hardware, firmware, and/or software that can be used to establish a Wi-Fi connection, a Bluetooth connection, a cellular connection, an Ethernet connection, and so on.

In some embodiments, memory 2010 can include any suitable storage device or devices that can be used to store instructions, values, data, or the like, that can be used, for example, by processor 2002 to present content using display 2004, to communicate with server 1952 via communications system(s) 2008, and so on. Memory 2010 can include any suitable volatile memory, non-volatile memory, storage, or any suitable combination thereof. For example, memory 2010 can include random-access memory (RAM), read-only memory (ROM), electrically programmable ROM (EPROM), electrically erasable ROM (EEPROM), other forms of volatile memory, other forms of non-volatile memory, one or more forms of semi-volatile memory, one or more flash drives, one or more hard disks, one or more solid state drives, one or more optical drives, and so on. In some embodiments, memory 2010 can have encoded thereon, or otherwise stored therein, a computer program for controlling operation of computing device 1950. In such embodiments, processor 2002 can execute at least a portion of the computer program to present content (e.g., images, user interfaces, graphics, tables), receive content from server 1952, transmit information to server 1952, and so on. For example, the processor 2002 and the memory 2010 can be configured to perform the methods described herein (e.g., the method illustrated in the workflow of FIG. 1; the methods illustrated in the workflows of FIGS. 2A-2E; the method of FIG. 3; the method illustrated in the workflow of FIG. 9; the method of FIG. 12; the method illustrated in the workflow of FIG. 16).

In some embodiments, server 1952 can include a processor 2012, a display 2014, one or more inputs 2016, one or more communications systems 2018, and/or memory 2020. In some embodiments, processor 2012 can be any suitable hardware processor or combination of processors, such as a CPU, a GPU, and so on. In some embodiments, display 2014 can include any suitable display devices, such as an LCD screen, LED display, OLED display, electrophoretic display, a computer monitor, a touchscreen, a television, and so on. In some embodiments, inputs 2016 can include any suitable input devices and/or sensors that can be used to receive user input, such as a keyboard, a mouse, a touchscreen, a microphone, and so on.

In some embodiments, communications systems 2018 can include any suitable hardware, firmware, and/or software for communicating information over communication network 1954 and/or any other suitable communication networks. For example, communications systems 2018 can include one or more transceivers, one or more communication chips and/or chip sets, and so on. In a more particular example, communications systems 2018 can include hardware, firmware, and/or software that can be used to establish a Wi-Fi connection, a Bluetooth connection, a cellular connection, an Ethernet connection, and so on.

In some embodiments, memory 2020 can include any suitable storage device or devices that can be used to store instructions, values, data, or the like, that can be used, for example, by processor 2012 to present content using display 2014, to communicate with one or more computing devices 1950, and so on. Memory 2020 can include any suitable volatile memory, non-volatile memory, storage, or any suitable combination thereof. For example, memory 2020 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 2020 can have encoded thereon a server program for controlling operation of server 1952. In such embodiments, processor 2012 can execute at least a portion of the server program to transmit information and/or content (e.g., data, images, a user interface) to one or more computing devices 1950, receive information and/or content from one or more computing devices 1950, receive instructions from one or more devices (e.g., a personal computer, a laptop computer, a tablet computer, a smartphone), and so on.

In some embodiments, the server 1952 is configured to perform the methods described in the present disclosure. For example, the processor 2012 and memory 2020 can be configured to perform the methods described herein (e.g., the method illustrated in the workflow of FIG. 1; the methods illustrated in the workflows of FIGS. 2A-2E; the method of FIG. 3; the method illustrated in the workflow of FIG. 9; the method of FIG. 12; the method illustrated in the workflow of FIG. 16).

In some embodiments, data source 1902 can include a processor 2022, one or more data acquisition systems 2024, one or more communications systems 2026, and/or memory 2028. In some embodiments, processor 2022 can be any suitable hardware processor or combination of processors, such as a CPU, a GPU, and so on. In some embodiments, the one or more data acquisition systems 2024 are generally configured to acquire data, images, or both, and can include an MRI system. Additionally or alternatively, in some embodiments, the one or more data acquisition systems 2024 can include any suitable hardware, firmware, and/or software for coupling to and/or controlling operations of an MRI system. In some embodiments, one or more portions of the data acquisition system(s) 2024 can be removable and/or replaceable.

Note that, although not shown, data source 1902 can include any suitable inputs and/or outputs. For example, data source 1902 can include input devices and/or sensors that can be used to receive user input, such as a keyboard, a mouse, a touchscreen, a microphone, a trackpad, a trackball, and so on. As another example, data source 1902 can include any suitable display devices, such as an LCD screen, an LED display, an OLED display, an electrophoretic display, a computer monitor, a touchscreen, a television, etc., one or more speakers, and so on.

In some embodiments, communications systems 2026 can include any suitable hardware, firmware, and/or software for communicating information to computing device 1950 (and, in some embodiments, over communication network 1954 and/or any other suitable communication networks). For example, communications systems 2026 can include one or more transceivers, one or more communication chips and/or chip sets, and so on. In a more particular example, communications systems 2026 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 2028 can include any suitable storage device or devices that can be used to store instructions, values, data, or the like, that can be used, for example, by processor 2022 to control the one or more data acquisition systems 2024, and/or receive data from the one or more data acquisition systems 2024; to generate images from data; present content (e.g., data, images, a user interface) using a display; communicate with one or more computing devices 1950; and so on. Memory 2028 can include any suitable volatile memory, non-volatile memory, storage, or any suitable combination thereof. For example, memory 2028 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 2028 can have encoded thereon, or otherwise stored therein, a program for controlling operation of data source 1902. In such embodiments, processor 2022 can execute at least a portion of the program to generate images, transmit information and/or content (e.g., data, images, a user interface) to one or more computing devices 1950, receive information and/or content from one or more computing devices 1950, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for reconstructing an image of a subject from k-space data acquired with a magnetic resonance imaging (MRI) system, the method comprising:
accessing k-space data with a computer system, wherein the k-space data were acquired from a subject using an MRI system, wherein the k-space data comprise timeframes of k-space data acquired using time-interleaved undersampling patterns in k-space;
generating composite image data from the k-space data using the computer system to combine timeframes of the k-space data;
accessing a machine learning model with the computer system, wherein the machine learning model has been trained on training data to extract ghosting artifact signal components from a magnetic resonance image;
generating a ghosting artifact image by inputting the composite image data to the machine learning model using the computer system, generating the ghosting artifact image as an output, wherein the ghosting artifact image depicts ghosting artifacts extracted from the composite image data;
estimating outer volume signals using the computer system to subtract the ghosting artifact image from the composite image data;
generating outer volume removed k-space data using the computer system to remove the outer volume signals from the k-space data; and
reconstructing an image from the outer volume removed k-space data using the computer system.

2. The method of claim 1, wherein the k-space data were acquired from a spatial region in the subject, wherein the spatial region contains a smaller region-of-interest containing an anatomical target.

3. The method of claim 2, wherein the anatomical target is a heart.

4. The method of claim 3, wherein the k-space data comprise timeframes of k-space data acquired during times in a cardiac cycle when the heart is moving.

5. The method of claim 3, wherein the k-space data comprise timeframes of k-space data acquired during times in a cardiac cycle when the heart is not moving.

6. The method of claim 1, wherein the image is reconstructed from the outer volume removed k-space data using a physics-driven deep learning (PD-DL) model by:
accessing the PD-DL model using the computer system; and
inputting the outer volume removed k-space data to the PD-DL model, generating the reconstructed image as an output.

7. The method of claim 6, wherein reconstructing the image using the PD-DL model further comprises accessing masked coil sensitivity maps with the computer system and inputting the masked coil sensitivity maps to the PD-DL model as an additional input.

8. The method of claim 7, wherein accessing the masked coil sensitivity maps with the computer system comprises:
accessing coil sensitivity maps with the computer system;
accessing an outer volume mask data with the computer system; and
generating the masked coil sensitivity maps by applying the outer volume mask data to the coil sensitivity maps.

9. The method of claim 8, wherein the outer volume mask data are generated from the estimated outer volume signals.

10. The method of claim 1, wherein the k-space data comprise timeframes of uniformly undersampled k-space.

11. The method of claim 1, wherein the k-space data comprise a higher density of k-space sampling in a first region of k-space and a lower density of k-space sampling in a second region of k-space.

12. The method of claim 11, wherein the ghosting artifacts comprise noise-like ghosting caused by differences between the k-space data acquired from the first region of k-space and the k-space data acquired from the second region of k-space.

13. The method of claim 1, wherein reconstructing the image comprises using an image reconstruction that receives coil sensitivity maps as an additional input.

14. The method of claim 13, wherein the coil sensitivity maps are generated from outer volume removed calibration data in which outer volume regions have been removed.

15. The method of claim 14, wherein the outer volume removed calibration data are generated by:
generating an outer volume mask from the estimated outer volume signals; and
applying the outer volume mask to the calibration data.

16. The method of claim 14, wherein the calibration data comprise the composite image data.

17. The method of claim 1, wherein reconstructing the image comprises reconstructing a time-series of images.

18. A method for generating outer volume removed calibration data for use in magnetic resonance imaging (MRI), the method comprising:
accessing k-space data with a computer system, wherein the k-space data were acquired from a subject using an MRI system, wherein the k-space data comprise timeframes of k-space data acquired using time-interleaved undersampling patterns in k-space;
generating composite image data from the k-space data using the computer system to combine timeframes of the k-space data;
estimating outer volume signals from the composite image data;
generating outer volume removed calibration data using the computer system to remove the outer volume signals from the composite image data; and
storing the outer volume removed data with the computer system.

19. The method of claim 18, further comprising generating coil sensitivity maps from the outer volume removed calibration data.

20. A method for reconstructing an image of a subject from k-space data acquired with a magnetic resonance imaging (MRI) system, the method comprising:
accessing k-space data with a computer system, wherein the k-space data were acquired from a heart of a subject with an MRI system using a pulse sequence, wherein the k-space data comprise at least one timeframe of k-space data that samples a central region of k-space and at least one timeframe of k-space data that samples peripheral regions of k-space during dead time of a pulse sequence during which the heart is moving;
reconstructing a first image from the k-space data using a deep learning image reconstruction that is trained on training data to remove image artifacts;
generating composite image data from the k-space data using the computer system to combine timeframes of the k-space data;
estimating outer volume signals using the computer system to subtract the first image from the composite image data;
generating outer volume removed k-space data using the computer system to remove the outer volume signals from the k-space data; and
reconstructing a second image from the outer volume removed k-space data using the computer system.

21. The method of claim 20, wherein the pulse sequence is an ECG-triggered pulse sequence that acquires auxiliary k-space data during an auxiliary window occurring after a primary imaging window during which the k-space data are acquired.

22. The method of claim 21, wherein the auxiliary k-space data sample peripheral k-space lines that are interleaved with the k-space data.

23. The method of claim 21, wherein the auxiliary data are further used to estimate the outer volume signals.
